(19) United States
(12) Patent Application Publication
(10) Pub. No.: US 2023/0332203 A1
(43) Pub. Date: Oct. 19, 2023

(54) RAPID ANTIBIOTIC SUSCEPTIBILITY TESTING

(71) Applicant: IOWA STATE UNIVERSITY RESEARCH FOUNDATION, INC., Ames, IA (US)

(72) Inventors: Young-Jin LEE, Ames, IA (US); Evan LARSON, Maplewood, MN (US); Josiah RENSNER, Ames, IA (US)

(21) Appl. No.: 18/300,640

(22) Filed: Apr. 14, 2023

Related U.S. Application Data
(60) Provisional application No. 63/331,434, filed on Apr. 15, 2022.

(51) Int. Cl. C12Q 1/18

(52) U.S. Cl. CPC C12Q 1/18

(57) ABSTRACT

An antimicrobial susceptibility test was developed that utilizes deuterium labeling of membrane lipids to track the growth of bacterial cells. Deuterium labeling of lipids can be detected using matrix-assisted laser desorption/ionization mass spectrometry. Additionally, bacteria growth is performed on the MALDI target, minimizing sample preparation materials and time. The labeling efficiency, or the ratio of labeled to unlabeled lipid peaks, provides information about the growth rate of bacteria. This growth ratio can differentiate between resistant and susceptible strains of bacteria as a resistant strain will maintain ˜50% labeling efficiency between untreated and treated cultures. In comparison, a susceptible strain will see a decrease in fractional abundance of deuterium from ˜50% in the untreated to ˜10% in the treated. This approach is applied to measure the minimum inhibitory concentration of the resistant and susceptible strains from on-target microdroplet culture in a range of antibiotic concentrations.

### RELATED APPLICATIONS

This application claims priority under 35 U.S.C. § 119(e) to U.S. Provisional Patent Application No. 63/331,434, filed Apr. 15, 2023, which is incorporated herein by reference.

### BACKGROUND OF THE INVENTION

Antimicrobial resistance and the emergence of multidrug resistant bacteria is a growing concern for human and veterinary healthcare. Antibiotic misuse and overuse have led to highly resistant bacterial strains, including ESKAPE (Enterococcus faecium, Staphylococcus aureus, Klebsiella pneumoniae, Acinetobacter baumannii, Pseudomonas aeruginosa, and Enterobacter) pathogens, which are dangerous and difficult to treat. As new antibiotic development slows, it is imperative to develop new methods to diagnose and determine treatment for infections quickly and accurately in a clinical setting.

Antibiotic susceptibility tests (AST) are commonly used in clinical settings to determine the minimum inhibitory concentration (MIC) of an antibiotic for a particular strain (Antibiotics 2022, 11, 427). The MIC provides the minimum concentration at which the antibiotic inhibits the growth of a strain of bacteria and gives a reference for the resistance of that strain. The most commonly used methods, such as broth dilution or agar dilutions, can provide MIC information regardless of resistance mechanism but suffer from long culture times, leading to a slow turn-around time (TAT) from diagnosis to treatment. Methods such as polymerase chain reaction (PCR) amplification of resistance genes or immunoassays can rapidly identify known resistance genes. These methods rely on databases that may not be comprehensive and provide no MIC information. The ideal clinical AST method should have a fast TAT, function regardless of the resistance mechanism or bacterial strain, and be simple and high-throughput.

Matrix assisted laser desorption/ionization-mass spectrometry (MALDI-MS) has become widely available in the clinical labs, especially MALDI-time of flight (TOF) systems such as the MALDI Biotyper or Vitek systems to identify bacterial strains. These MALDI-TOF systems are high-throughput with short analysis time and high sensitivity, allowing for rapid identification of species or strains based on their protein fingerprint. MALDI-MS has been recently used for AMR detection beyond bacterial identification. Idelevich et al. (J Clin Microbiol 2018, 56 (10), e00913) developed an AST using on-target microdroplet culture of bacteria to detect cell growth by protein fingerprinting, which successfully identified resistant strains from agar cultures and directly from blood cultures. The Goodlett (Sci Rep 2020, 10 (1), 21536) and Ernst (Sci Rep 2018, 8 (1), 15857) groups have developed on-target extraction and detection of membrane glycolipids, specifically Lipid A, to classify gram negative bacterial strains and identify colistin resistance. Additionally, they have cataloged membrane glycolipids to fingerprint ESKAPE pathogens and their resistance (Anal Chem 2019, 91 (2), 1286). Zhang et al. (Anal. Chem. 2018, 90 (6), 3863) identified metabolic biomarkers to identify extended-spectrum β-lactamase-resistant E. coli strains. Other studies have focused on detecting hydrolysis products of β-lactam drugs such as carbapenem, which can be detected very quickly (Int J Antimicrob Agents 2016, 48 (6), 655). Currently, there is no clinical method that takes advantage of the high-throughput and sensitivity of MALDI-MS to detect antimicrobial resistance and determine the MIC of a strain.

It is challenging to develop a rapid AST typically limited by the culture time in response to antibiotics. Some automatic tools are developed based on microscopic physiological changes and approved by the Food and Drug Administration, but they still require 4-16 hours of turn-around time. Stable isotope labeling can rapidly track metabolic changes as organisms grow. Berry et al. tracked the incorporation of deuterium labels using Raman spectroscopy to quickly identify and sort bacteria in a mouse cecum sample (PNAS 2015, 112 (2), E194). Tao et al. used Raman spectroscopy to track deuterium incorporation to determine MIC (Anal. Chem. 2017, 89 (7), 4108). Kopf et al. tracked pathogen growth rate in cystic fibrosis patients using isotope ratio mass spectrometry (Proc Natl Acad Sci U S A 2016, 113 (2), E110). Neubauer et al. developed an extraction method for deuterium labeled lipids and used deuterium incorporation to measure bacterial growth (Rapid Commun Mass Spectrom 2018, 32 (24), 2129).

A major problem is that existing antibiotic susceptibility tests are slow and do not take full advantage of current mass spectrometry techniques. Accordingly, there is a need for a high-throughput method that can yield antibiotic susceptibility results in less time than traditional methods.

### SUMMARY

Incorporation of a deuterium label (“D-label”) into newly synthesized lipids can be easily detected by mass spectrometry and by using this method, one can determine if bacteria are growing, dividing, and producing new lipids. Additionally, deuterium labeling is a straightforward technique and simply growing bacteria in the presence of D₂O allows for the rapid detection of newly synthesized lipids.

Herein we disclose using stable isotope labeling of membrane lipids, specifically deuterium labeling, to track the growth of bacterial strains in the presence of antibiotics using MALDI-MS to determine resistance or MIC. We combine it with the on-target microdroplet culture method to simplify the sample preparation by culturing and preparing the sample for MALDI analysis on a single plate. However, instead of typing bacteria using protein signals, which requires a minimum of four hours, we measure a minute amount of deuterium labeling in membrane lipids, which dramatically reduces the culture time. As a proof-of-concept, a model E. coli system was used with two strains, JJ1886 and MG1655, which are resistant and susceptible to ciprofloxacin, respectively. It is also demonstrated on clinically relevant S. aureus species, methicillin-resistant Staphylococcus aureus (MRSA), using MALDI Biotyper.

Accordingly, this disclosure provides a method for determining antibiotic susceptibility comprising:
a) incubating an untreated mixture and at least one treated mixture, wherein:
   each mixture comprises a bacterial culture and deuterium oxide (D₂O), wherein the bacterial culture comprises a D₂O concentration of about 5% v/v to about 50% v/v;
   the treated mixture comprises an antibiotic of a set concentration, whereas the untreated mixture lacks an antibiotic; and
   the incubation is for a period of time sufficient for the untreated mixture to produce deuterium-labeled membrane lipids;
b) drying the untreated mixture and the treated mixture to provide a dry untreated mixture and a dry treated mixture, each comprising bacteria from the bacterial culture;
c) lysing the bacteria in each dry mixture to release deuterium-labeled membrane lipids that comprise deuterium atoms bonded to carbon atoms of the membrane lipids, if present, thereby providing a lysed untreated mixture and a lysed treated mixture;
d) contacting the lysed untreated mixture and the lysed treated mixture with a protic solvent, thereby exchanging deuterium atoms bonded to heteroatoms of the deuterium-labeled membrane lipids with hydrogen atoms from the protic solvent;
e) removing the protic solvent from the lysed untreated mixture and the lysed treated mixture to provide an untreated sample and a treated sample for analysis, wherein soluble contaminants are removed with the protic solvent; and
f) analyzing the untreated sample and the treated sample for deuterium incorporation by mass spectrometry, thereby determining the amount of deuterium incorporation in the samples; and comparing the deuterium incorporation in the untreated sample to the deuterium incorporation in the treated sample; thereby determining the antibiotic susceptibility of the bacterial culture. In some embodiments, one or more of steps a)-f) can be omitted and/or one or more elements of the recited method can be omitted. Furthermore, other techniques and limitations described herein can be included in the method, in various other embodiments.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Sample preparation workflow for deuterium labeling AST experiment. Workflow parameters such as D₂O concentration and culture time can be adjusted as needed.

FIG. 2A-C. (a) Mass spectrum of PE 32:1 after labeling with 20% deuterium for two hours. Two main distributions of peaks are present, unlabeled “old lipids” present at the start of the culture and labeled “new lipids” newly synthesized in on-target microdroplet culture. * indicates matrix or broth background. (b) Isotopologue distribution for PE 32:1 after two-hour cultures for a range of D₂O concentrations (0%-35%). The labeled peak envelope is shifted based on the D₂O concentration. (c) The plot for the average molecular weight of PE 32:1 vs. D₂O concentration.

FIG. 3. Fractional abundance of deuterium calculated for five common membrane lipids in JJ (resistant) strain in 20% D₂O after two hours. D-Labeling Efficiency is calculated from only new lipids (M+3−M+13).

FIG. 4A-B. (a) Average molecular weight of PE 32:1 at different time points for both JJ (resistant) and MG (susceptible) strains of E. coli cultured in 20% D₂O with or without 10 μg/mL ciprofloxacin at three different time points. (b) Labeling efficiency for the same cultures as mentioned above, comparing treated vs. untreated for resistant and susceptible.

FIG. 5A-B. (a) Average molecular weight of PE 32:1 for JJ (resistant) and MG (susceptible) after two-hour culture with 20% D₂O in a range of ciprofloxacin concentrations. (b) Average molecular weight of PE 32:1 for MG after two-hour culture with 20% D₂O in a narrower range of ciprofloxacin concentrations. MIC values measured by the traditional broth dilution method are indicated in the figure as a circle and matching with the MIC values measured by the current method defined as the concentration where average molecular weight is significantly reduced due to the decrease in deuterium labeling.

FIG. 6. Comparison of the average molecular weight of PE 32:1 for MG (susceptible) from the deuterium labeling experiment vs. the cell count obtained from a traditional broth dilution experiment. The decrease in cell count and the decrease in labeling correlate well over a range of ciprofloxacin concentrations.

FIG. 7A-D. Isotope distribution for four common (a-d) labeled lipids for JJ (resistant) strain in 20% D₂O after two hours.

FIG. 8A-B. Isotope distribution for MG (susceptible) (a) and JJ (resistant) (b) across a range of ciprofloxacin concentrations in 20% D₂O in two hours.

FIG. 9A-B. MALDI-MS of S. aureus grown in a 20% D₂O culture obtained by Orbitrap (a) and Biotyper (b) for the analysis of PG species in negative ion mode using N-naphthylethylenediamine dihydrochloride (NEDC) as matrix. Multiple PGs were observed with D-labeling by both the mass spectrometers.

FIG. 10A-B. Average mass of PG 32:0 in susceptible and resistant S. aureus treated with a range of methicillin concentrations and grown in a 20% D₂O culture determined using Orbitrap (a) and Biotyper (b). This data demonstrates the applicability of the proposed AST for clinically relevant MRSA using Biotyper.

### DETAILED DESCRIPTION

Rapid antibiotic susceptibility testing (AST) is a pressing need in veterinary and human health care. Current methods of AST are greatly limited by turn-around-time leading to slower treatment of serious infections. Common AST methods rely on direct growth response in the presence of antibiotics (broth or agar dilution methods), or growth response in the presence of antibiotics measured by protein abundance (MALDI-TOF). Each of these methods require a longer culture (4-16 hours) to detect a cellular response and determine susceptibility. Genotypic methods such as PCR or laminar flow immunoassays are much faster but are targeted, require known resistance genes or antibodies and provide no information on the minimum inhibitory concentration (MIC). Development of a rapid untargeted AST method that can be easily implemented into clinical settings is a necessary step to further combat resistant bacteria.

To accomplish this, a MALDI-mass spectrometry method is used to rapidly identify metabolic responses to antibiotics which can be used to determine susceptibility. We utilize the rapid metabolic response of bacteria to determine susceptibility in as little as half an hour using MALDI-MS. Metabolic response is much faster than cell growth response and can provide additional information about resistance mechanisms while also measuring MIC. Culturing bacteria in the presence of deuterium labeled water can label any newly formed metabolites, especially lipids, as cells grow or replicate. When culturing in the presence of D₂O as well as an antibiotic, any labeled lipids detected indicate active bacterial metabolism and growth and a resistance to that antibiotic. The percent of the labeled lipids vs. total lipids provides a quantitative measure of cell growth which can be directly related to MIC by culturing with a range of antibiotic concentrations.

Preliminary data for E. coli resistant to ciprofloxacin have shown successful labeling of phosphatidylethanolamine lipids with D₂O. Deuterium labelling of lipids can happen very quickly, even in the lag phase of a new culture, as cells grow and produce new lipids. Based on data obtained, labelling of membrane phospholipids can be detected in a culture as short as 30 minutes. Also, it was successfully demonstrated on clinically relevant S. aureus species using MALDI Biotyper. These results show a potential to identify metabolic markers of resistance as well as isotopically label newly synthesized lipids for growth measurement in half hour cultures.

Additional information and data supporting the invention can be found in the following publication by the inventors: Journal of the American Society for Mass Spectrometry 2022, 33 (7), 1221-1228 and its Supporting Information, which are incorporated herein by reference in its entirety.

### Definitions.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001, and Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991).

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “heteroatom” refers to any atom in the periodic table that is not carbon or hydrogen. Typically, a heteroatom is O, S, N, P. The heteroatom may also be a halogen, metal or metalloid.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound.

The term “IC₅₀” is generally defined as the concentration required to inhibit a specific biological or biochemical function by half, or to kill 50% of the cells in a designated time period, typically 24 hours.

As used herein, the term “minimum inhibitory concentration” or “MIC” refers to the lowest concentration of an antimicrobial (for example, an antibiotic) drug that will inhibit the visible growth of a microorganism after an incubation period.

General laboratory techniques (DNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

### Embodiments of the Technology.

This disclosure provides a method for determining antibiotic susceptibility comprising:
a) incubating an untreated mixture and at least one treated mixture, wherein:
   each mixture comprises a bacterial culture and deuterium oxide (D₂O), wherein the bacterial culture comprises a D₂O concentration of about 5% v/v to about 50% v/v;
   the treated mixture comprises an antibiotic of a set concentration, whereas the untreated mixture lacks an antibiotic; and
   the incubation is for a period of time sufficient for the untreated mixture to produce deuterium-labeled membrane lipids;
b) drying the untreated mixture and the treated mixture to provide a dry untreated mixture and a dry treated mixture, each comprising bacteria from the bacterial culture;
c) lysing the bacteria in each dry mixture to release deuterium-labeled membrane lipids that comprise deuterium atoms bonded to carbon atoms of the membrane lipids, if present, thereby providing a lysed untreated mixture and a lysed treated mixture;
d) contacting the lysed untreated mixture and the lysed treated mixture with a protic solvent, thereby exchanging deuterium atoms bonded to heteroatoms of the deuterium-labeled membrane lipids with hydrogen atoms from the protic solvent;
e) removing the protic solvent from the lysed untreated mixture and the lysed treated mixture to provide an untreated sample and a treated sample for analysis, wherein soluble contaminants are removed with the protic solvent; and
f) analyzing the untreated sample and the treated sample for deuterium incorporation by mass spectrometry, thereby determining the amount of deuterium incorporation in the samples; and comparing the deuterium incorporation in the untreated sample to the deuterium incorporation in the treated sample; thereby determining the antibiotic susceptibility of the bacterial culture.

In some embodiments, the volume (in μL) of the bacterial culture and the volume (in μL) of D₂O are each independently 0.1, 0.5, 1, 2, 3, 4, 5, 6, 7, 8, 9, 10, or more. In some embodiments, the volume of the bacterial culture and the volume of D₂O are the same or about the same (e.g., ±10-20%). In some other embodiments, the volume of the bacterial culture is 1 μL to 3 μL and the volume corresponds in a 1:1 ratio with the volume of D₂O, i.e., 1 μL to 3 μL, respectively. In various embodiments, a mixture of the bacterial culture and D₂O includes a drug, while in other embodiments, the mixture does not include a drug. In embodiments that include a drug, the drug can be an antibiotic.

In one embodiment, the method for determining antibiotic susceptibility comprises:
a) incubating an untreated mixture comprising deuterium oxide (D₂O) and a bacterial culture having a set concentration of bacteria, wherein the untreated mixture lacks an antibiotic;
b) incubating at least one treated mixture comprising an antibiotic, D₂O and the bacterial culture;
c) drying the untreated mixture and the at least one treated mixture;
d) lysing the bacterial culture in the dry untreated and dry treated mixture thereby releasing deuterium labeled membrane lipids;
e) contacting the lysed untreated mixture and lysed treated mixture with a protic solvent, wherein exchangeable deuterium atoms that are bonded to heteroatoms when present in the labeled lipids are exchanged to hydrogen atoms;
f) removing the protic solvent from the lysed untreated mixture and lysed treated mixture to provide an untreated and treated sample for analysis, wherein soluble contaminants are removed with the protic solvent;
g) analyzing for an amount of deuterium incorporation in the untreated and treated sample by mass spectrometry; and
h) comparing the amount of deuterium incorporation in the untreated sample to the treated sample;
   wherein the bacterial culture comprises a D₂O concentration of about 5% v/v to about 50% v/v; and the concentration of the antibiotic is established for each of the at least one treated mixture; wherein the susceptibility of the antibiotic is determined.

In various embodiments, the concentration or amount of the antibiotic, and/or D₂O, and/or bacteria in the culture in each mixture is known or has been determined by standard methods. In various embodiments, the D₂O concentration in the bacterial culture is about 10% v/v to about 40% v/v. In various embodiments, the concentration of D₂O by vol/vol (v/v) is about 5%, about 10%, about 15%, about 20%, about 25%, about 30%, about 35%, about 40%, about 50%, about 60%, about 70% or about 80%. In some embodiments, the D₂O is neat D₂O that has been diluted to a concentration of less than 70% (v/v). In other embodiments, the neat D₂O is diluted with H₂O, a broth culture, or culture medium.

In some embodiments, the untreated mixture is at least one untreated mixture or one or more untreated mixtures.

In various embodiments, the deuterium-labeled membrane lipids comprise deuterium-labeled phosphatidylethanolamines (PE), deuterium-labeled phosphatidylglycerols (PG), cardiolipin (CL) or a combination thereof.

In various embodiments, the deuterium-labeled membrane lipids comprise deuterium-labeled PE 30:0, PE 32:1, PE 33:1, PE 34:1, or PG 34:1. In one specific embodiment, the deuterium-labeled membrane lipids comprise deuterium-labeled PE 32:1.

In various embodiments, the untreated mixture and the at least one treated mixture are incubated in one chamber, or separate chambers for each of the mixtures (the untreated mixture and the at least one treated mixture each comprise D₂O in the appropriate ratio by volume described herein), wherein the one chamber or each of the separate chambers is humidified with a volume of D₂O that is about the same concentration of D₂O that is in the untreated mixture and/or the at least one treated mixture. For example, the bottom of each chamber would comprise a volume of D₂O to maintain an atmosphere of D₂O in the chamber such that mixtures are not affected by water vapor exchange.

In various embodiments, the untreated mixture and the at least one treated mixture are incubated for about 0.25 hours to about 4 hours. In some embodiments, the untreated mixture and the at least one treated mixture are incubated for about 0.25 hours, 0.5 hours, about 1 hour, about 1.5 hours, about 2 hours, about 2.5 hours, about 3 hours, about 3.5 hours, or about 4 hours.

In some embodiments, the concentration of the bacterial culture is about 0.1×10⁸ CFU/mL to about 10×10⁸ CFU/mL. In various embodiments, the concentration of the bacterial culture in CFU/mL is about 1×10⁸, about 2×10⁸, about 3×10⁸, about 4×10⁸, or about 5×10⁸. In further embodiments, the concentration of the bacterial culture in CFU/mL is about 1×10⁶ to about 1×10⁷, about 1×10⁷ to about 1×10⁸, or about 1×10⁸ to about 1×10⁹.

In some embodiments, the method comprises determining that the bacteria are resistant to the antibiotic at a fixed concentration when the amount of deuterium incorporated in the untreated sample is about the same as the treated sample. In other embodiments, the method comprises determining that the bacteria are susceptible to the antibiotic at the fixed concentration when the amount of deuterium incorporated in the untreated sample is more than the treated sample.

In some embodiments, the bacteria comprise one or more of Enterococcus faecium, Staphylococcus aureus, Klebsiella pneumoniae, Acinetobacter baumannii, Pseudomonas aeruginosa, and Enterobacter (ESKAPE pathogens).

In various embodiments, lysing the bacteria comprises contacting the dry mixture with an alcohol and drying the resulting mixture. In additional embodiments, removing the protic solvent comprises wicking or blotting the protic solvent. In further embodiments, the protic solvent comprises (or is) an aqueous acid or trifluoroacetic acid. In yet additional embodiments, the untreated sample and treated sample are contacted with a MALDI matrix compound, such as an alcoholic solution of an organic acid, a benzoic acid, a dihydroxybenzoic acid (DHB), or N-(1-naphthyl) ethylenediamine dihydrochloride (NEDC), prior to analysis by mass spectrometry (MS). In some embodiments, DHB is used for positive ion mode MS. In other embodiments, NEDC is used for negative ion mode MS.

In some embodiments, the method separately comprises incubating the at least one treated mixture with the antibiotic at a plurality of different concentrations. In various embodiments, each of the at least one treated mixture (or untreated mixture) comprises at least one different bacterial concentration. In additional embodiments, the method comprises determining the minimum inhibitory concentration (MIC) of the antibiotic by any standard method that is known to a person of ordinary skill in the art.

Also, this disclosure provides a method for analyzing deuterium uptake in a bacterial culture comprising:
a) incubating a first mixture of deuterium oxide (D₂O) and a bacterial culture at a set concentration;
b) drying the mixture;
c) lysing the bacterial culture in the dry mixture thereby releasing membrane lipids comprising deuterium-labeled lipids to provide a lysed mixture and unlabeled natural lipids;
d) contacting the lysed mixture and a protic solvent or an aqueous acid, thereby exchanging deuterium atoms bonded to heteroatoms, when present in the labeled lipids, with hydrogen atoms from the protic solvent or aqueous acid;
e) removing the protic solvent or aqueous acid from the lysed mixture to provide a sample for analysis, wherein soluble contaminants are removed with the protic solvent or aqueous acid; and
f) analyzing for an amount of deuterium incorporation in the untreated and treated sample by mass spectrometry; or analyzing for a ratio of deuterium-labeled lipids and unlabeled natural lipids in the sample by mass spectrometry;
wherein the bacterial culture comprises a D₂O concentration of about 5% v/v to about 50% v/v;
the first mixture is incubated for about 0.25 hours to about 2 to 4 hours in a chamber comprising a solution that has about the same concentration of D₂O as the concentration of D₂O in the bacterial culture; and the membrane lipids are PE 30:0, PE 32:1, PE 33:1, PE 34:1, or PG 34:1.

In various embodiments, the first mixture further comprises an antibiotic and the concentration of the antibiotic in the mixture is established.

### Results and Discussion.

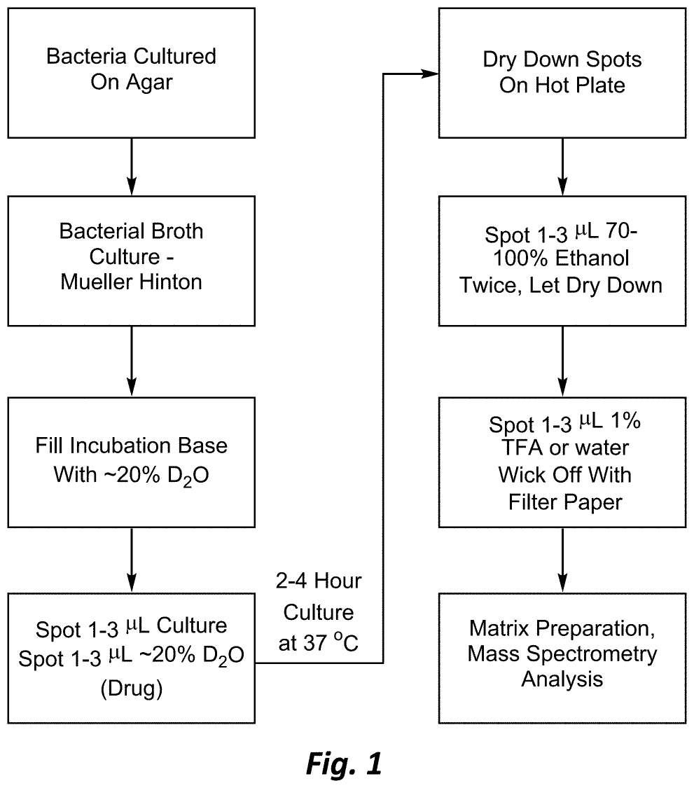

Method Development. The adapted microdroplet method is shown in FIG. 1. After overnight culture on an agar plate, the bacteria are scooped into a broth culture and adjusted to 2×10⁸ CFU/mL. A higher initial bacteria concentration was used in this work compared to the Idelevich work (1×10⁶ CFU/mL) as this approach focuses on detecting a minute amount of deuterium labeling in lipids with a minimum culture time. This broth culture is then spotted in 3 μL droplets on a stainless-steel microarray plate, and 3 μL of D₂O with or without ciprofloxacin is spotted on top. The plate is then cultured at 37° C. in a plastic incubation chamber adapted from a Bruker Biotyper plate transport container. The bottom part of the chamber contains 4 mL of D₂O to maintain a humid atmosphere, which guarantees the droplet does not dry out during incubation. It is important to ensure that the D₂O concentration in the base of the chamber matches the concentration on the droplet, so the droplet concentration is not affected by water vapor exchange.

After a 30 minute to two-hour culture, the culture droplets are dried using a hot plate. This allows the cell lipids to adhere to the plate and not be lost in the subsequent steps. After drying down, 3 μL of ethanol are spotted twice to lyse cells and free membrane lipids and the spots are allowed to air dry. After ethanol treatment, 3 μL of 1% TFA is spotted and wicked off using filter paper after 30 seconds. This removes most Mueller Hinton broth interferences while leaving behind lipids. These two treatments were optimized based on lipid signals. After treatment, matrix is applied, and the plate is analyzed on the mass spectrometer.

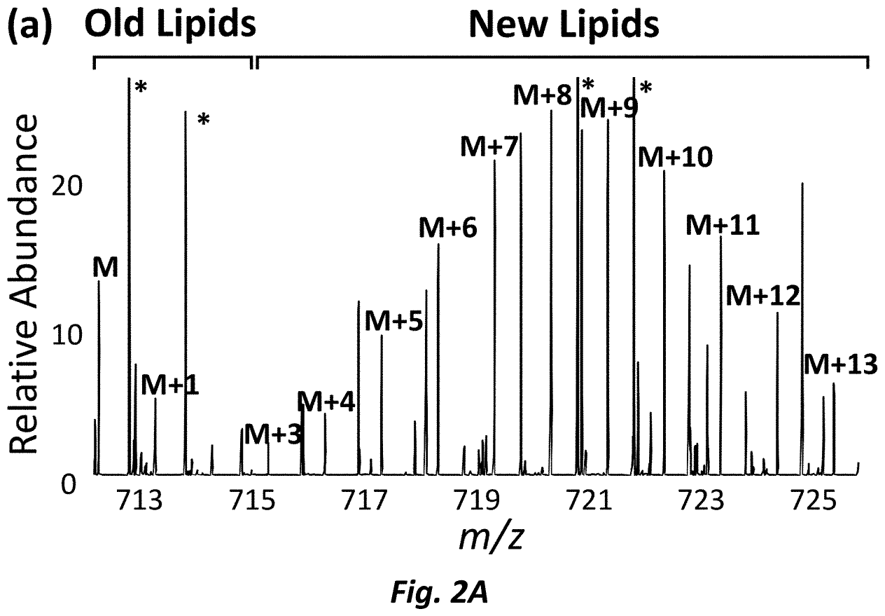

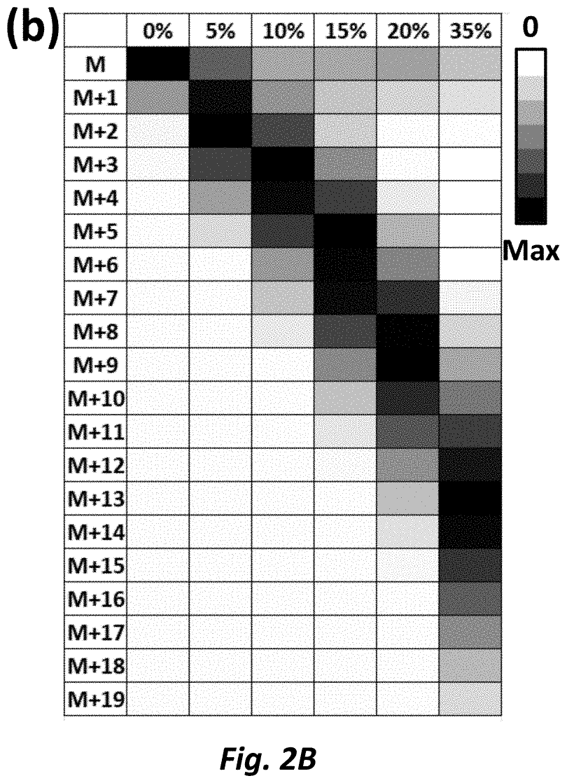

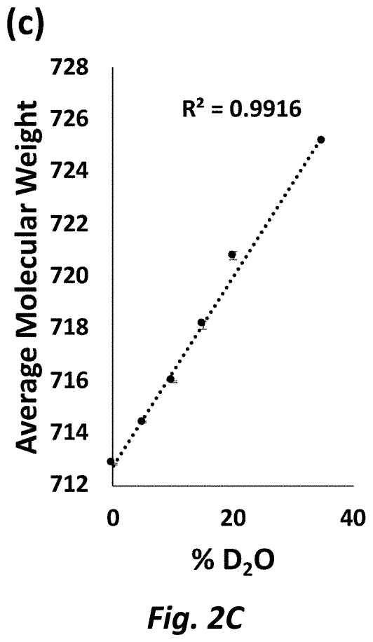

Typical labeled data is shown in FIG. 2A for PE 32:1, a common E. coli membrane lipid, after 2-hour culture. With 20% D₂O, the lipid peaks are separated into two isotope distributions, “old” lipids and “new” lipids. The “old” lipids consist of the unlabeled monoisotopic peak and single ¹³C peak with the natural ¹³C abundance. These peaks represent the lipids of bacterial cells present at the start of the culture. The new lipids consist of a Gaussian distribution of deuterium labeled peaks, typically centered around M+7 or M+8 for 20% D₂O. These peaks represent deuterium labeling of the fatty acyl chains of the phospholipid, which in turn is a metric for new growth in the microdroplet culture.

The 1% TFA wash and the matrix application provide opportunities for the back exchange of readily exchangeable hydrogens (e.g., —OH) so that only carbon bound deuterium will be measured, which improves the reproducibility of the data. The ratio between the new lipids (labeled) and the old lipids (unlabeled) provides a metric for the lipid turnover rate and therefore the growth and division of the bacteria in the microdroplet. Alternatively, calculating the average molecular weight of a particular lipid in different conditions provides a simple method for comparing lipid growth in those conditions, such as D₂O concentration, antibiotic concentration, or differing strains. For example, in three replicates for 20% D₂O labeling, the average molecular weight of PE 32:1 is 720.7843 Da compared to an unlabeled control which is 712.8373 Da.

D₂O Concentration Optimization and Lipid Selection. D₂O is lethal to complex organisms at about 20-40% of body water content, but bacteria are amenable to higher concentrations, allowing for tunability of the labeled peaks. As shown in FIG. 2B, deuterium labeling of PE 32:1 is readily observed for a broad range of D₂O concentrations. Adjusting the D₂O concentration shifts the position of the Gaussian peak distribution and the average molecular weight of the lipid. The linear correlation between D₂O concentration and average molecular weight (FIG. 2C) suggests no detrimental effect to E. coli at least up to 35% D₂O concentration. At 5% D₂O, some of the labeled and unlabeled peaks (e.g., natural ¹³C₁-PE 32:1 vs. in vivo labeled ²H₁-PE 32:1) are nearly entirely overlapped due to the insufficient mass resolution and difficult to differentiate. At 35% D₂O, in contrast, the labeled peaks are clearly distinguishable from old lipids but widely distributed.

For a complete separation and high deuterium labeling, 20% D₂O was chosen for subsequent experiments but any of 10-35% D₂O could be used. The capability of shifting these labeled peak distributions to higher or lower m/z provides a means to avoid overlapping background peaks or other lipids. This would be especially useful when applying this method to lower resolution instruments, such as the MALDI Biotyper, to detect labeling from overlapping interferences. Although the washing alleviates signal suppression issues, there are still background interferences in the current work (e.g., labeled with ‘*’ in FIG. 2A). This is not a concern with the current instrumentation as we have sufficient mass resolution to resolve lipids and contamination peaks. However, they can be further removed with additional washing or different work-up process for MALDI Biotyper if necessary.

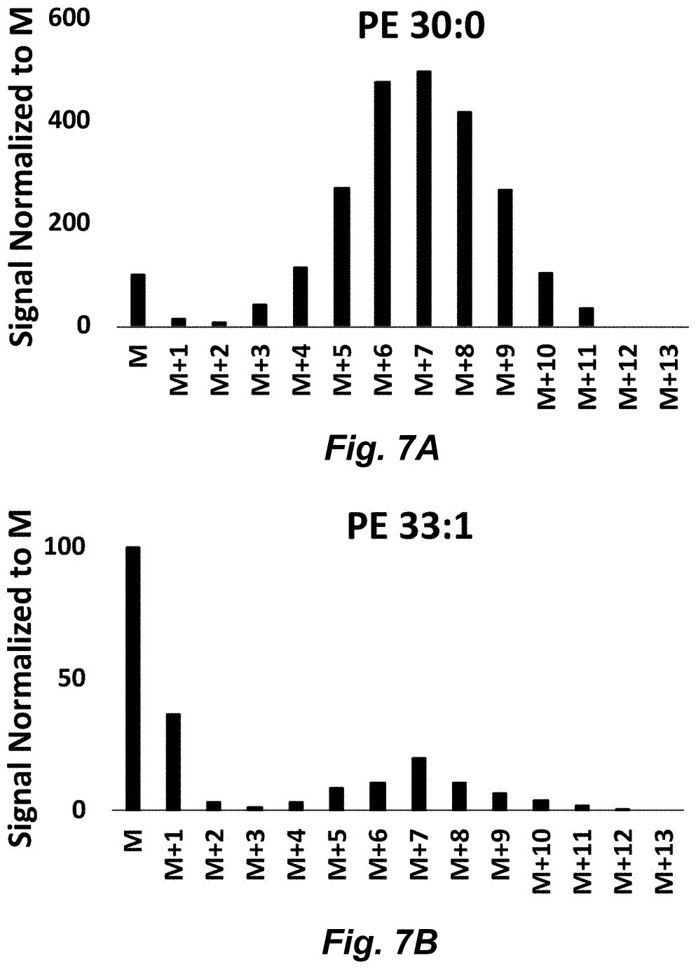

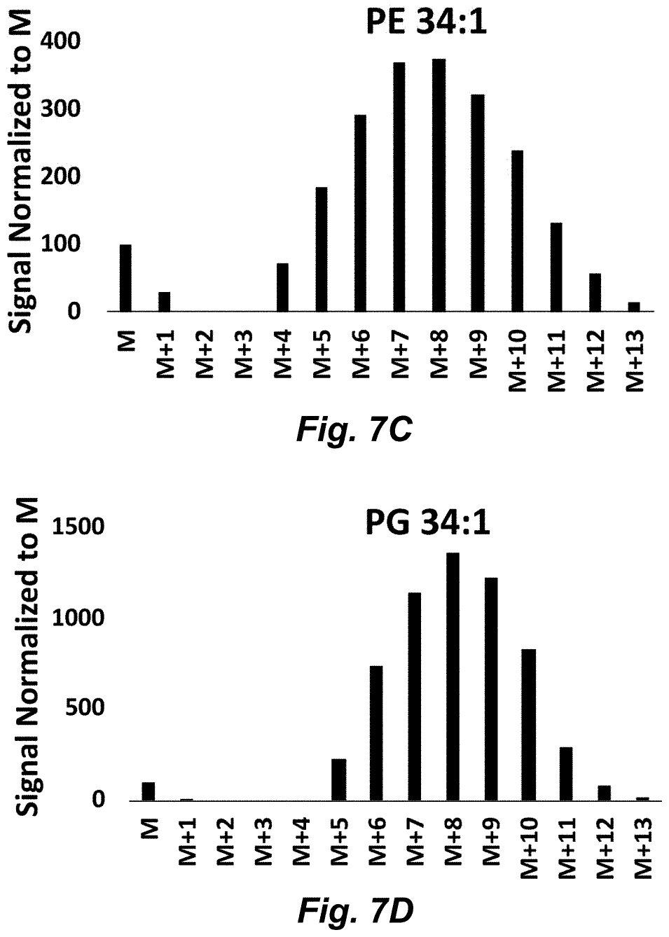

In addition to PE 32:1, the most abundant membrane lipid, efficient deuterium labeling is detected for all other lipids (FIG. 7). The labeled peaks are well separated from the unlabeled peak region (M˜M+2). The number of possible deuterium labeling can be calculated as the total number of carbon-bound hydrogens multiplied by D₂O concentration. For example, PE 32:1 has 69 C-bound hydrogens and 20% of them, 13.8, could be labeled by deuterium. However, the kinetic isotope effect makes in vivo deuterium labeling less efficient than that of hydrogen, mainly in the biosynthesis of nicotinamide adenine dinucleotide phosphate (NADPH) and fatty acids. The efficiency of deuterium labeling can be calculated as the average number of labeled deuterium out of the theoretical number of deuterium labeling. For example, the average of ˜7.6 D-labeling in PE 32:1 for newly synthesized lipids corresponds to —55% fractional abundance of deuterium (F_{D-label}).

Most mass spectrometers do not have sufficient mass resolution to resolve deuterium vs. ¹³C-isotope in the lipid mass range, including the current work. Alternatively, the FD-label can be calculated from the average molecular weight (Equation 1),

$${F}_{D - label} = \frac{( {MW}_{D 2 O} - {MW}_{H 2 O} ) / ( {m}_{D} - {m}_{H} )}{( Number of {H}_{C - bound} ) \times ( {D}_{2} O conc . )} \qquad ( Equation 1 )$$

where MW_{D2O} and MW_{H2O} represent the measured average molecular weight of the lipid species in D₂O vs. H₂O medium, respectively, and (m_{D}−m_{H}) represent the mass difference between deuterium and hydrogen atom mass, 1.006277 Da. Equation 1 is convenient because it is applicable even when there is no clear separation between new and old lipids (e.g., 5% D₂O in FIG. 2B). Additionally, it corrects contribution from the natural isotopes, including D-labeling of ¹³C₁-lipids. However, it is distinguished from D-labeling efficiency in that it accounts for the deuterium abundance out of the total lipids including both old and new lipids.

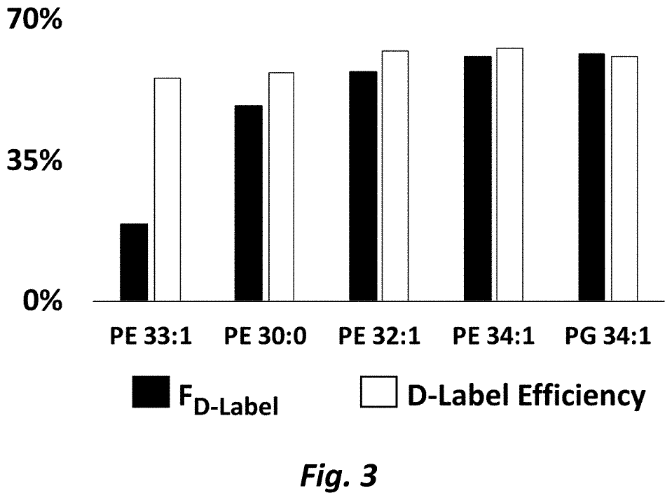

As shown in FIG. 3, the fractional abundance of deuterium varies for different lipid species. PE 30:0, PE 32:1, and PE 34:1 have F_{D-label} of 45-60% with the M+7 and M+8 peaks ˜3.5-5× higher than the monoisotopic peak (FIG. 7, FIG. 2a). PG 34:1 has the highest F_{D-label} of ˜61% with M+8 peak roughly 14× higher than the monoisotopic peak. In contrast, PE 33:1 still has a significant amount of old lipids (M˜M+2) compared to new lipids with only ˜19% of F_{D-label}. However, the D-labeling efficiency calculated from the new lipids alone (FIG. 3, D-Label Efficiency, i.e., from M+3 through M+13) is ˜50-60% and still comparable to that of other lipids (50˜60%), suggesting it is due to the lipid composition change in newly synthesized cell membranes. It is well known that lipid composition changes depending on the phase of bacterial growth (Mass Spectrom 2018, 32 (24), 2129).

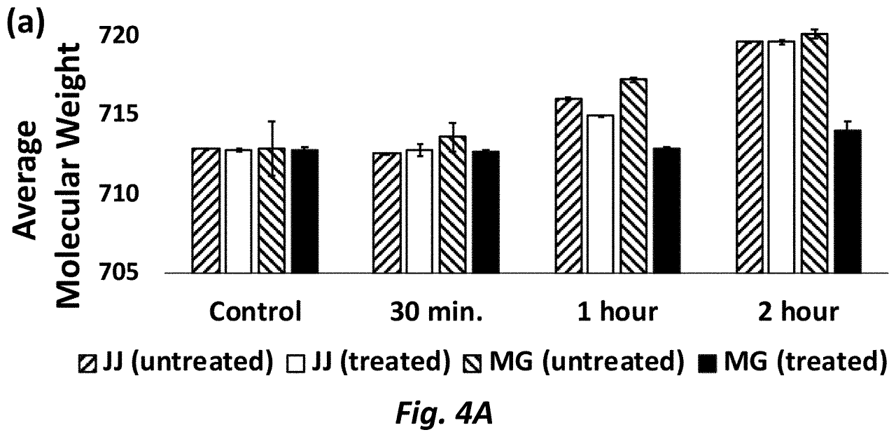

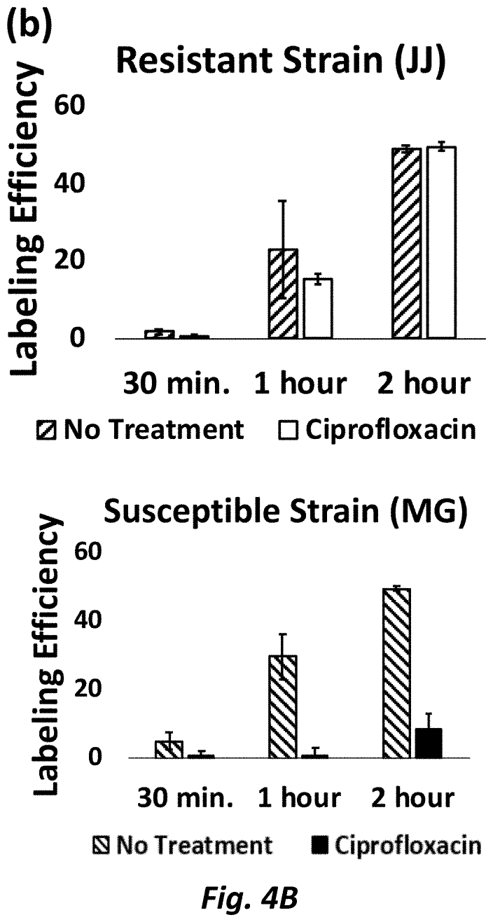

Antibiotic Treatment and Minimum Inhibitory Concentration. The next step is testing the membrane lipid labeling response to antibiotic treatment. Based on previous experiments, PE 32:1 at 20% D₂O was used for the rest of the experiments. FIG. 4A shows the average molecular weight of PE 32:1 after culturing in 20% D₂O, or H₂O for the control, at multiple time points with or without 10 μg/mL ciprofloxacin. The 30-minute D₂O culture for 30 minutes are almost indistinguishable from two hour H₂O culture because the labeled peaks in the 30-minute culture are present but in very low abundance compared to the unlabeled monoisotopic peak. It is mainly attributed to the slow growth in the lag phase.

As the culture time increases to one or two hours, the average molecular weight of the lipid increases for both strains when not exposed to ciprofloxacin. However, a contrast is observed for the antibiotic treated cultures as the average molecular weight increases for the resistant strain (JJ) but stays relatively consistent for the susceptible (MG). This is expected as an effective antibiotic will halt the growth and replication of susceptible bacterial cells, stopping the uptake and incorporation of deuterium in lipid cell membrane biosynthesis. The growth rate is initially slightly slower for the resistant strain in the 1-hour data, especially with antibiotic treatment as they respond to antibiotics; however, the difference becomes negligible in 2-hour data compared to the susceptible strain without treatment. FIG. 4B shows the labeling efficiency for each time point with and without treatment. As expected, the discrepancy in labeling is most prominent in the two-hour culture for the susceptible strain while the treated resistant is comparable to the untreated.

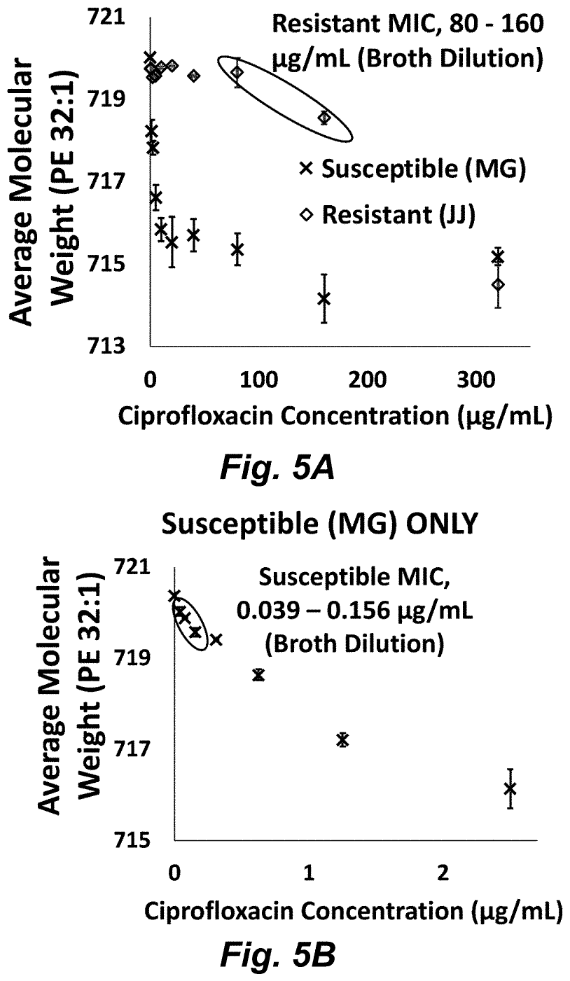

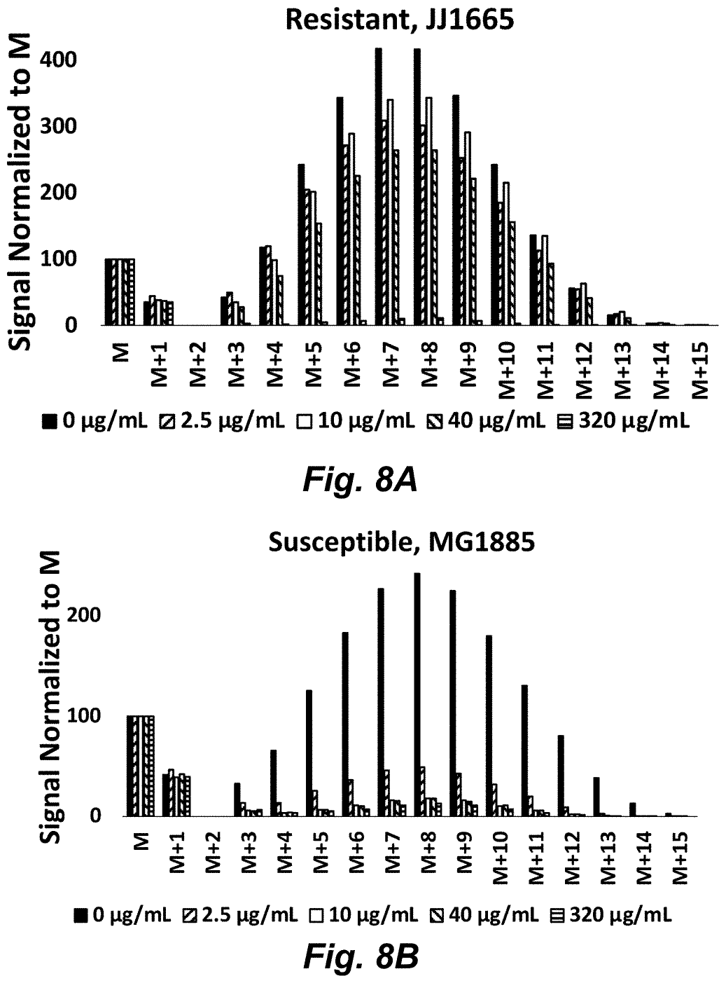

Finally, an MIC study was performed by MALDI-MS of on-target microdroplet culture with in vivo deuterium labeling. FIG. 5A shows the average molecular weight of PE 32:1 after a two-hour culture with 20% D₂O for a range of ciprofloxacin concentrations. The isotope distributions are extracted from the mass spectral data are also shown in FIG. 8. The molecular weight of the lipid remains consistent for the resistant strain up to an extreme antibiotic concentration, 80 μg/mL, but at higher concentrations, bacteria cannot process the amount of antibiotic present and are inhibited, resulting in a decrease in the molecular weight. On the other hand, the susceptible strain is inhibited at the first antibiotic concentration as a decrease in labeling is immediately observed. The molecular weight decreases to a plateau across a broad range of concentrations (10 μg/mL to 320 μg/mL). This is expected as the lowest concentration (1.25 μg/mL) is still far above previously recorded MICs for MG1655 obtained from broth dilution, 0.078 μg/mL, so bacterial growth is strongly inhibited.

An additional experiment was performed with a lower range of antibiotic concentrations to determine the MIC of susceptible MG1655 strain (FIG. 5B). A decrease in labeling is observed even for the smallest drug concentration, 0.039 μg/mL, confirming that ciprofloxacin effectively inhibits the strain. Additionally, this highlights that the method is sensitive enough to detect slight decreases in labeling (<0.5 Da) at low antibiotic concentrations. For both resistant and susceptible strains, the average molecular weight at the very high concentration does not decrease to that of control (714.5˜715.2 Da vs. 712.8 Da). This is because some newly labeled lipids are produced before cell death.

This data on its own is useful in determining resistant and susceptible strains as the decrease in labeling indicates antibiotic efficacy. However, a comparison to a traditional MIC measurement is necessary to quantitatively validate the deuterium labeling MIC for its potential clinical use. To accomplish this, a broth dilution experiment was used to determine the concentration of ciprofloxacin which inhibits the resistant and susceptible strains. This method takes about three days total and requires manual counting of the colonies on the agar plate. The approximate concentration where the number of colonies decreases compared to the untreated control indicates the MIC. The MIC determined by this broth dilution method is indicated in FIG. 5 to compare with the deuterium labeling MIC.

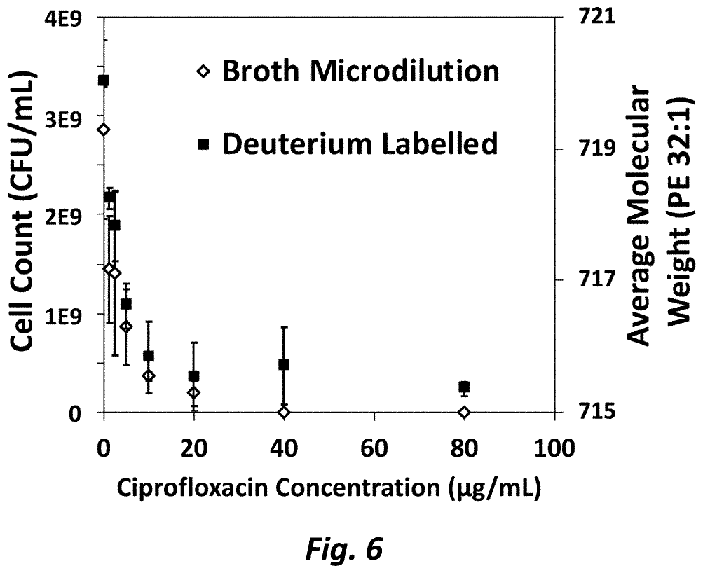

The traditional MIC for the resistant strain is between 80-160 μg/mL ciprofloxacin, which corresponds to the first point in the deuterium labeling MIC where the labeling starts to decrease. The susceptible strain has a much lower MIC range between 0.039 and 0.156 μg/mL but also corresponds to the decrease in the deuterium labeling. In addition, the overall cell count from the broth dilution at each concentration also corresponds well to the deuterium labeling method (FIG. 6). These results indicate this method can not only differentiate between resistant and susceptible strains but can also be directly related to the MIC value for each strain.

Conclusion. In this work, we developed an on-target microdroplet culturing method with D₂O to label bacterial membrane lipids and track bacterial growth. When culturing with a range of antibiotics, the resistance of a strain can be determined by the F_{D-label} or average molecular weight of common membrane lipids, such as PE 32:1 in E. coli. In addition, the MIC can be approximated by the first antibiotic concentration at which the labeling efficiency begins to decrease, which gives comparable MIC values obtained by traditional broth dilution method. Typical MIC experiments have slow TATs, while the deuterium labeling experiment takes only one or two hours to see the clear resistance. Furthermore, MALDI-MS data acquisition can be very fast as little as a few minutes per plate, which means that multiple strains, antibiotics, or antibiotic concentrations can be cultured and analyzed in a single high throughput experiment. The material demands of the experiment are also much lower than traditional methods as only a small volume of bacteria culture and antibiotic/D₂O are necessary for each culture. Overall, the deuterium labeling method proposed here provides a high throughput, a low material method to determine MIC, and, more broadly, the resistance or susceptibility of a particular strain of bacteria. This approach should be applicable regardless of antibiotic, bacterial strain, or resistance mechanism.

Furthermore, this work can be used for studying other bacteria and antibiotic combinations. E. coli are fast-growing, gram-negative bacteria. Other bacteria may take longer to culture and produce enough labeled signals; however, their growth should be detected faster than other traditional growth methods. Different species and strains will have different lipid distributions, and background peaks may overlap the most abundant labeled lipids. The deuterium concentration can be adjusted to shift the lipid distributions to avoid contamination. The general workflow will remain the same and the adaption to new bacteria and antibiotics could be easily made. Finally, applying the method to more popular mass spectrometers in clinical labs, such as a Bruker Biotyper, can bring it to broader applications. As the Biotyper has a much lower resolution than the current Orbitrap instrumentation, with appropriate adjustments, the labeled peaks should be detectable, especially when compared to the unlabeled spectrum.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Experimental Methods

Microdroplet Bacterial Culture. Two Escherichia coli strains, MG1655 (MG) and JJ1886 (JJ), were removed from −80° C. storage and streaked onto a Columbia blood agar plate. The initial culture was made by incubating the agar plates overnight in a 37° C. incubator to grow the initial bacteria stock. After overnight incubation, each bacteria strain was inoculated into 1 mL of Mueller Hinton broth (Sigma Aldrich-USA or Thermo Fisher Scientific). A colony-forming unit (CFU) value was determined for the initial stock using an OD600 measurement, and then the stock was diluted to a concentration of 2×10⁸ CFU/mL.

The base of the incubation chamber (Transport Box for MSP Biotarget, 8270006, Bruker Daltonics) was filled with 4 mL of D₂O adjusted to the concentration used in the growth medium, typically 20%. A μFocus array plate (Hudson Surface Technology, Closter, NJ) was cut in half and placed into the incubation chamber. A 3 μL volume of the broth culture of each strain was spotted onto the plate, and then 3 μL of D₂O was spotted on top of that, with or without ciprofloxacin. The final concentration of D₂O on the spot was equivalent to the concentration in the base of the chamber. Each condition was spotted in triplicate on the plate, typically alternating between the two strains. After spotting, the plates were transferred to the incubator at 37° C. for 30 minutes, one hour or two hours, depending on the experiment. After incubation, each plate was placed onto a hotplate (−100° C.) and dried down until each culture spot was completely dry. To lyse the cells, 3 μL of ethanol was spotted onto each culture twice and allowed to air dry between and after each application. Afterward, 3 μL of 1% trifluoroacetic acid (TFA) was spotted on each culture. After 30 seconds, the TFA was wicked off using filter paper which removed the majority of the water-soluble broth contamination. Bacteria experiments were performed in a biosafety level 2 lab, and bacteria on the MALDI plates were inactivated using methods approved by the Institutional Biosafety Committee (IBC).

Broth Dilution Experiment for Minimum Inhibitory Concentration. After overnight culture on Columbia blood agar plates, both E. coli strains were inoculated into Mueller Hinton broth at a concentration of 2×10⁸ CFU/mL. A 50 μL volume of each strain culture were then mixed with 50 μL of a range of ciprofloxacin concentrations (0, 1.25, 2.5, 5, 10, 20, 40, 80, 160, 320 μg/mL for MG and 40, 80, 160, 320 μg/mL for JJ) in 20% D₂O and cultured overnight. After overnight culture, each culture was then diluted 10-fold seven times (10⁰, 10⁻¹, 10⁻², 10⁻³, 10⁻⁴, 10⁻⁵, 10⁻⁶, 10⁻⁷). A 20 μL aliquot of each dilution was spotted in triplicate on Colombia blood agar plates and cultured overnight. After overnight culture, the number of colonies formed for each dilution was counted and converted to CFU/mL. An additional broth dilution experiment was performed just for MG with a ciprofloxacin concentration range of (0, 0.039, 0.078, 0.156, 0.313, 0.625, 1.25, 2.5 μg/mL).

Mass Spectrometry Analysis and Data Analysis. After lysing and cleaning steps, 3 μL of 30 mg/mL 2,5-dihydroxybenzoic acid (DHB) in 70:30 MeOH:0.1% TFA was spotted onto each culture and dried down. Plates were analyzed on a MALDI source (MALDI Injector; Spectroglyph, Kennewick, WA) coupled to an Orbitrap mass spectrometer (QExactive HF; Thermo Fisher Scientific, San Jose, CA). The MALDI source uses a 349 nm laser (Explorer One, Spectra Physics, Milpitas, CA) at 500 Hz repetition rate with an energy of ˜4 μJ. Data were collected in positive mode for m/z range of 680-850 with 120,000 resolution at m/z 200 in profile mode. Once collected, ASCII data were extracted using QualBrowser (Thermo) from raw files. An in-house Python script was used to extract labeled lipid peaks and calculate the average molecular weight for each lipid.

### Example 2. Summary of Experimental Steps

We developed an AST method that can be performed in 30 minutes to 2 hours; works regardless of the resistance mechanism; and wherein the culture, sample preparation, and analysis (on a single slide) are simplified. In this work, the AST method entails these features by detecting deuterium labelling of bacterial membrane lipids using MALDI-MS. The disclosed method can be exemplified as follows:
a) Two E. coli strains, MG 1655 which is susceptible to ciprofloxacin, and JJ 1886 which is resistant to ciprofloxacin, were cultured on agar plates overnight;
b) Each culture was scraped off the agar plate, suspended in 1 mL of Mueller Hinton Broth, and the concentration was adjusted to 2×10⁸ CFU/mL;
c) 3 μL of each culture were spotted in three replicates onto μarray plate (Hudson Surface Technologies) and 3 μL of D₂O (w/ or w/o ciprofloxacin) was spotted on top;
d) The spotted plate was placed into the incubation chamber with 4 mL of D₂O matching the on-spot concentration in the base and cultured at 37° C. for 30 minutes to 2 hours;
e) After culturing, the plate was dried down using a hotplate, 3 μL of ethanol was spotted twice and dried down to lyse cells, then 3 μL of 1% TFA was spotted and wicked off using filter paper to remove broth contaminants;
f) After washings steps, 3 μL of 30 mg/mL 2,5-dihydroxybenzoic acid in ethanol was spotted on each washed culture;
g) Plates were run on a Spectroglyph MALDI source coupled to a QExactive HF in positive mode at m/z 680-850; and
h) Python script was written to analyze data.

### Example 3. Comparison of Analytical Methods

The disclosed method was tested using the Bruker Biotyper and the results were compared to data using the method that was previously established on an Orbitrap mass spectrometer. A common cell membrane lipid, phosphatidylglycerol (PG) 32:0 was used as an indicator. Since a higher average mass is indicative of more growth, a reduced average mass indicates susceptibility to methicillin. The Biotyper is an economical and more accessible instrument but has less resolution and more error in the measurements. However, data from the Biotyper results in the same conclusions when compared to data from the Orbitrap.

For the resistant strain, the additional of methicillin does not decrease the average mass significantly, but a constant decrease for the susceptible strain was found with increasing methicillin concentration. The exact masses calculated from both instrument are slightly different due to the increased error in the Biotyper measurements, but both instruments follow the same trend and the Biotyper data can still clearly distinguish the two strains.

### Experimental Details.

Staphylococcus aureus 25923 and methicillin-resistant Staphylococcus aureus 33591 were cultured in a 37° C. incubator on agar plates. Cells were then suspended in cation-adjusted Mueller-Hinton broth and diluted to a concentration of 8×10⁸ cfu/mL using a OD600 measurement and a correction factor of 0.1=5×10⁸ cfu/mL. Samples were prepared on SGT μFocus plates (Hudson Surface Technology) and a reusable MSP BigAnchor 96 BC MALDI target plate (Bruker). Three μL of the broth culture and 3 μL of 40% D₂O with varying methicillin concentrations were spotted onto the μFocus plates, while 1.5 μL each of broth culture and 40% D₂O solutions were spotted onto the BigAnchor plate. This results in final cultures in 0.5×cation-adjusted Mueller-Hinton broth with 20% D₂O and an 4×10⁸ cfu/mL initial bacterial concentration with a total volume of 6 μL for μFocus samples and 3 μL for BigAnchor samples. Samples were placed in incubation chambers modified from Bruker MALDI target sample holders with 4 mL of 20% D₂O in the base and were sealed by wrapping with Parafilm M (Bemis). Samples were cultured in a 37° C. incubator for 4 hours.

After incubation samples were dried using a hot plate. The samples were removed from heat and then two treatments of 70% ethanol were spotted unto the samples to lyse the cells, allowing the samples to completely dry between applications. After the ethanol had dried, water was added and then after 30 seconds was wicked off using filter paper (Whatman Grade 1). 3 μL of ethanol and water was used for the μFocus plates and 1 μL of ethanol and 1.5 μL of water was used for the BigAnchor plates. N-(1-naphthyl) ethylenediamine dihydrochloride matrix (NEDC) was used for analysis. 10 mg/mL solutions of NEDC were prepared in methanol and 3 μL was spotted onto the μFocus samples and 1 μL was spotted onto the BigAnchor samples.

μFocus samples were analyzed in negative ion mode using an Orbitrap Q Exactive HF mass spectrometer (Thermo Scientific, San Jose, CA) with a Spectroglyph MALDI source (Spectroglyph, Kennewick, WA) equipped with a 349 nm laser (Explorer One; Spectra Physics, Milpitas, CA) using a 500 Hz repetition rate and ˜4 μJ per shot. Samples were analyzed at 120,000 resolving power at m/z 200. Spots were analyzed using 60 μm spatial resolution to ensure a good representation of the sample and resulting spectra were averaged in Xcalibur. Masslists from averaged spectra were then exported to .csv files and average mass of membrane lipids were calculated using a custom Python script. Samples were prepared in triplicate and the resulting average masses were averaged.

BigAnchor samples were analyzed using flexControl in negative ion mode using a Biotyper sirius MALDI-TOF mass spectrometer (Bruker, Billerica, MA). The LN_PepMix default method for analyzing peptides was used with the following voltage parameters: Ion Source 1=−10 kV, Ion Source 2=−9 kV, Lens=−3 kV. Default Bruker SmartBeam parameters were used, with 200 Hz and approximately 80% laser power. Laser power was varied from spot to spot to get an optimal signal to noise ratio. A 160 ns pulsed ion extraction time was used, with a mass range of 600-800 Da. All other parameters were instrument defaults: Digital Sensitivity (Full Scale)=100 mV, Linear Analog Offset=−3.1 mV, Detector Linear Base Gain Voltage=2500 V, Detector Linear Boost Gain Voltage=58 V.

Resulting spectra were opened using MSConvertGUI¹ and converted to mzXML files. The mzXMLs were then opened using mMass² Version 5.5.0. Batch processing peak picking was done with a signal/noise threshold of 1, an intensity threshold of 0, and a picking height of 100. This ensured that all peaks were automatically selected and m/z and intensity were copied into Excel. Data was externally calibrated using the monoisotopic mass of PG 32:0, and the average mass of PG 32:0 was calculated. Samples were prepared in quintuplicate and the resulting average masses were averaged.

As a comparison to the described mass spectrometry method, a traditional minimum inhibitory concentration (MIC) was also measured. Broth cultures of the two S. aureus strains were grown overnight at 37° C. in cation-adjusted Mueller Hinton Broth. An OD600 measurement was taken and the concentration was adjusted to OD600=0.5. A 1/100 dilution was then made in cation-adjusted Mueller Hinton broth. Fifty μL of this dilution was then pipetted into wells of a 96-well plate, and 50 μL of methicillin solutions at twice the desired final concentration was then added. This was done in triplicate for multiple methicillin concentrations as well as an untreated control for both strains and the plate was cultured overnight at 37° C. OD600 measurements were then taken and the optical density for averaged for each condition. A decrease of optical density indicated inhibition of growth and this data was used to calculate an MIC.

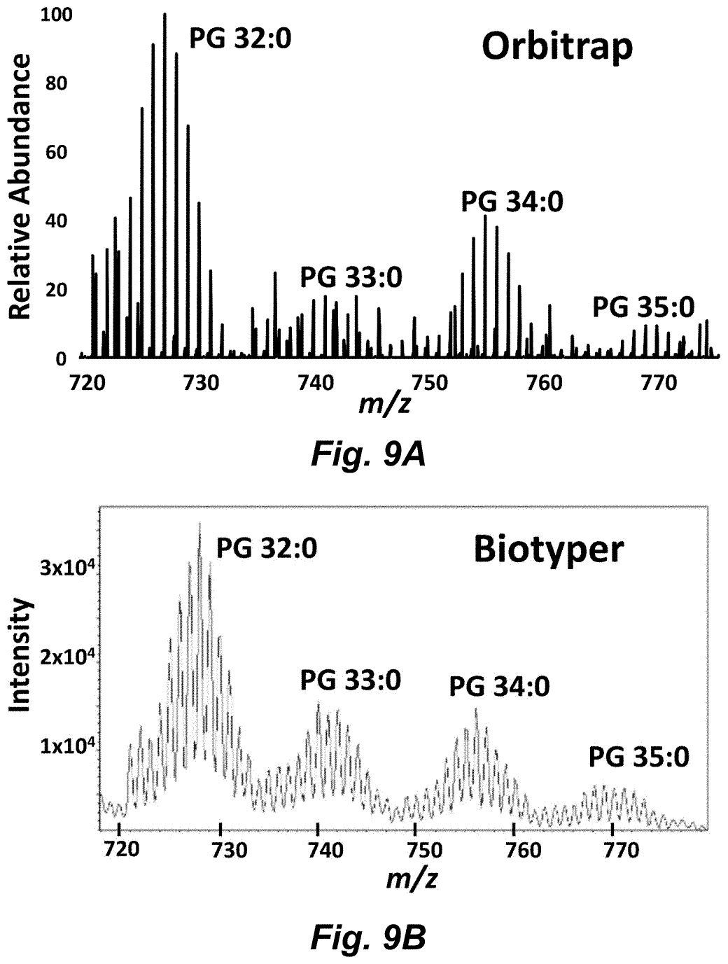

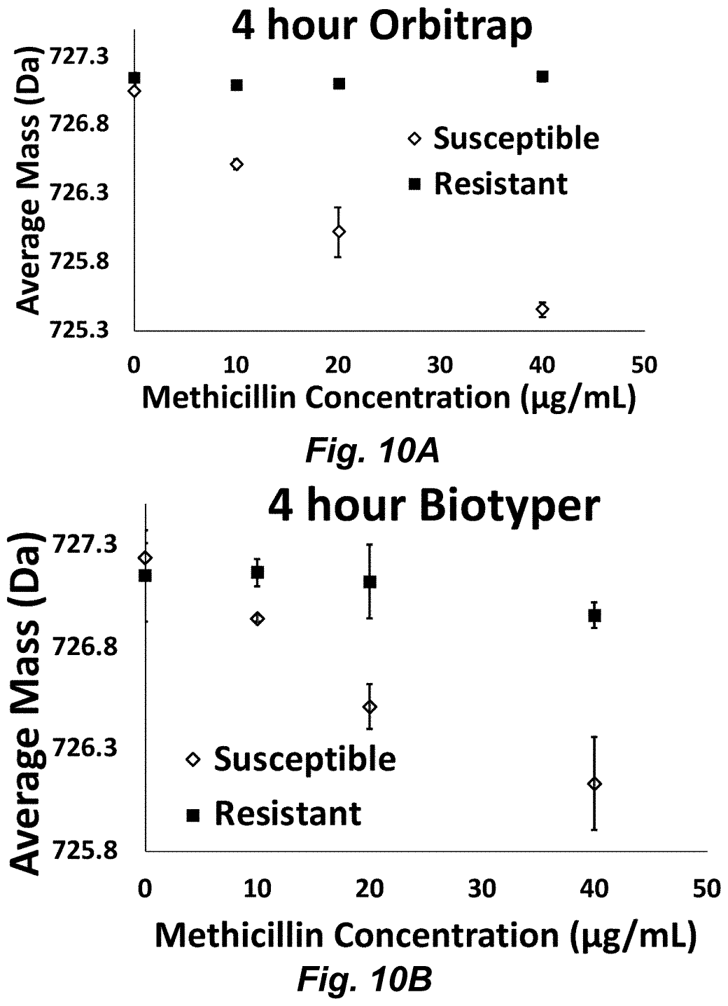

Average mass of treated and untreated samples are calculated and compared, with a lower average mass indicating less growth and susceptibility to the drug (FIG. 9 and FIG. 10). An MIC was calculated from each mass spectrometer for both the susceptible and resistant strain and then compared to the traditional approach.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for determining antibiotic susceptibility comprising:
a) incubating an untreated mixture and a treated mixture, wherein:
each mixture comprises a bacterial culture and deuterium oxide (D₂O), wherein the bacterial culture comprises a D₂O concentration of about 5% v/v to about 50% v/v;
the treated mixture comprises an antibiotic and the untreated mixture lacks an antibiotic; and
the incubation is for a period of time sufficient for the untreated mixture to produce deuterium-labeled membrane lipids;
b) drying the untreated mixture and the treated mixture to provide a dry untreated mixture and a dry treated mixture, each comprising bacteria from the bacterial culture;
c) lysing the bacteria in each dry mixture to release deuterium-labeled membrane lipids that comprise deuterium atoms bonded to carbon atoms of the membrane lipids, if present, thereby providing a lysed untreated mixture and a lysed treated mixture;
d) contacting the lysed untreated mixture and the lysed treated mixture with a protic solvent, thereby exchanging deuterium atoms bonded to heteroatoms of the deuterium-labeled membrane lipids with hydrogen atoms from the protic solvent;
e) removing the protic solvent from the lysed untreated mixture and the lysed treated mixture to provide an untreated sample and a treated sample for analysis; and
f) analyzing the untreated sample and the treated sample for deuterium incorporation by mass spectrometry, thereby determining the amount of deuterium incorporation in the samples; and comparing the deuterium incorporation in the untreated sample to the deuterium incorporation in the treated sample;
thereby determining the antibiotic susceptibility of the bacterial culture.

2. The method of claim 1 wherein the D₂O concentration in the bacterial culture is about 10% v/v to about 40% v/v.

3. The method of claim 2 wherein the D₂O concentration in the bacterial culture is about 20% v/v.

4. The method of claim 1 wherein the deuterium-labeled membrane lipids comprise deuterium-labeled phosphatidylethanolamines (PE), deuterium-labeled phosphatidylglycerols (PG), cardiolipin (CL) or a combination thereof.

5. The method of claim 4 wherein the deuterium-labeled membrane lipids comprise deuterium-labeled PE 30:0, PE 32:1, PE 33:1, PE 34:1, or PG 34:1.

6. The method of claim 5 wherein the deuterium-labeled membrane lipids comprise deuterium-labeled PE 32:1.

7. The method of claim 1 wherein the untreated mixture and the treated mixture are incubated in a chamber comprising a solution that has about the same concentration of D₂O as the concentration of D₂O in the untreated mixture and the treated mixture.

8. The method of claim 1 wherein the untreated mixture and the treated mixture are incubated for about 0.25 hours to about 4 hours.

9. The method of claim 1 wherein the bacterial culture has a concentration of about 0.1×10⁸ CFU/mL to about 10×10⁸ CFU/mL.

10. The method of claim 1 comprising determining that the bacteria are resistant to the antibiotic when the amount of deuterium incorporated in the untreated sample is about the same as the treated sample.

11. The method of claim 1 comprising determining that the bacteria are susceptible to the antibiotic when the amount of deuterium incorporated in the untreated sample is more than the treated sample.

12. The method of claim 1 wherein the bacteria comprise one or more of Enterococcus faecium, Staphylococcus aureus, Klebsiella pneumoniae, Acinetobacter baumannii, Pseudomonas aeruginosa, and Enterobacter (ESKAPE pathogens).

13. The method of claim 1 wherein lysing the bacteria comprises contacting the dry mixture with an alcohol and drying the resulting mixture.

14. The method of claim 1 wherein removing the protic solvent comprises wicking or blotting the protic solvent.

15. The method of claim 1 wherein the protic solvent comprises an aqueous acid.

16. The method of claim 1 wherein the untreated sample and treated sample are contacted with a MALDI matrix compound prior to analysis by mass spectrometry.

17. The method of claim 1 wherein the method comprises incubating more than one treated mixture with the antibiotic at a plurality of different concentrations.

18. The method of claim 17 further comprising determining the minimum inhibitory concentration (MIC) of the antibiotic.

19. A method for analyzing deuterium uptake in a bacterial culture comprising:
a) incubating a first mixture of deuterium oxide (D₂O) and a bacterial culture, wherein the bacterial culture comprises a D₂O concentration of about 5% v/v to about 50% v/v, and the first mixture is incubated for about 0.25 hours to about 4 hours in a chamber comprising a solution that has about the same concentration of D₂O as the concentration of D₂O in the bacterial culture;
b) drying the first mixture to provide a dry mixture;
c) lysing bacteria of the bacterial culture in the dry mixture to provide a lysed mixture of membrane lipids comprising deuterium-labeled lipids and unlabeled natural lipids, wherein the membrane lipids comprise PE 30:0, PE 32:1, PE 33:1, PE 34:1, PG 34:1, or a combination thereof;
d) contacting the lysed mixture and a protic solvent, thereby exchanging deuterium atoms bonded to heteroatoms, when deuterium labeled lipids are present, with hydrogen atoms from the protic solvent;
e) removing the protic solvent from the lysed mixture to provide a sample for analysis; and
f) analyzing for an amount of deuterium incorporation in the untreated and treated sample by mass spectrometry.

20. The method of claim 19 wherein the first mixture comprises an antibiotic.

21. A method for determining antibiotic susceptibility comprising:
a) incubating an untreated mixture comprising deuterium oxide (D₂O) and a bacterial culture having a fixed concentration of bacteria, wherein the untreated mixture lacks an antibiotic;
b) incubating a treated mixture comprising an antibiotic, D₂O, and a bacterial culture having the same fixed concentration of bacteria as the untreated mixture;
wherein the bacterial cultures comprise a D₂O concentration of about 5% v/v to about 50% v/v;
c) drying the untreated mixture to provide a dry untreated mixture, and drying the treated mixture to provide a dry treated mixture;
d) lysing bacteria of the bacterial culture in the dry untreated mixture and dry treated mixture to provide lysed mixtures of membrane lipids;
e) contacting the lysed untreated mixture and a protic solvent, and contacting the lysed treated mixture and a protic solvent;
f) removing the protic solvent from the lysed untreated mixture and lysed treated mixture to provide an untreated and a treated sample for analysis, wherein soluble contaminants are removed with the protic solvent;
g) analyzing for an amount of deuterium incorporation in the untreated and treated sample by mass spectrometry; and
h) comparing the amount of deuterium incorporation in the untreated sample to the amount of deuterium incorporation in the treated sample;
wherein the susceptibility of the antibiotic is thereby determined.
